(19) United States
(12) Patent Application Publication
(10) Pub. No.: US 2025/0090385 A1
(43) Pub. Date: Mar. 20, 2025

(54) ENDOLUMINAL TREATMENT DEVICES AND RELATED METHODS

(71) Applicant: Boston Scientific Scimed, Inc., Maple Grove, MN (US)

(72) Inventors: Rahul PRABHU, Brighton, MA (US); Jonathan ROOT, Townsend, MA (US); Martyn G. FOLAN, Co. Galway (IE)

(73) Assignee: Boston Scientific Scimed, Inc., Maple Grove, MN (US)

(21) Appl. No.: 18/890,230

(22) Filed: Sep. 19, 2024

Related U.S. Application Data
(60) Provisional application No. 63/583,969, filed on Sep. 20, 2023.

(51) Int. Cl. A61F 13/20; A61F 13/05

(52) U.S. Cl. CPC A61F 13/2071; A61F 13/05; A61F 13/204; A61F 2013/2014

(57) ABSTRACT

Medical devices and related methods useful for EVAC procedures are described. The medical device may include a sponge. The sponge may include a plurality of perforations that form one or more tear surfaces. The one or more tear surfaces may define a predefined location for removing a first portion of the sponge from a second portion of the sponge. The one or more tear surfaces may form a boundary between a radially inner surface of the first portion and a radially outer surface of the second portion. The medical device may further include a tube coupled to a proximal portion of the sponge.

### CROSS-REFERENCE TO RELATED APPLICATIONS

This application claims the benefit of priority to U.S. Provisional Application No. 63/583,969, filed on Sep. 20, 2023, which is incorporated by reference herein in its entirety.

### TECHNICAL FIELD

Various aspects of this disclosure relate generally to minimally invasive medical devices and methods. In particular, aspects of the disclosure relate to medical devices and methods for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the GI tract. Patients with perforations, post-surgical leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical devices of the current disclosure may rectify some of the deficiencies described above or address other aspects of the art.

### SUMMARY

Each of the aspects disclosed herein may include one or more of the features described in connection with any of the other disclosed aspects.

The disclosure includes medical devices and related methods useful for EVAC procedures. For example, the disclosure includes a medical device comprising a sponge. The sponge may include a plurality of perforations that form one or more tear surfaces. The one or more tear surfaces may define a predefined location for removing a first portion of the sponge from a second portion of the sponge. The one or more tear surfaces may form a boundary between a radially inner surface of the first portion and a radially outer surface of the second portion. The medical device may further comprise a tube coupled to a proximal portion of the sponge. The sponge may include an outer portion and an inner portion. The one or more tear surfaces may separate the outer portion from the inner portion. The outer portion may have a different shape from the inner portion. The inner portion may include at least one of the one or more tear surfaces. The at least one of the one or more tear surfaces of the inner portion may form one or more detachable layers of the inner portion. The one or more tear surfaces may be configured to define a plurality of sizes of an implantable portion of the sponge. The one or more tear surfaces may define a grid.

The one or more detachable layers may include two detachable layers. An outer surface of a first detachable layer of the two detachable layers may be adjacent to an inner surface of a second detachable layer of the two detachable layers. The first detachable layer may be concentric with the second detachable layer. The inner portion may further includes a core. The first detachable layer may be adjacent to the core.

In some examples, the outer portion may include a proximal surface. A proximal portion of at least one of the one or more detachable layers may extend proximally of the proximal surface of the outer portion. The proximal portion of the at least one of the one or more detachable layers may contact the tube.

In other examples, the outer portion may have a first surface and a second surface, and wherein at least one of the one or more tear surfaces extends from the first surface to the second surface between the inner portion and the outer portion. The outer portion may include at least one of the one or more tear surfaces. The at least one tear surface of the outer portion may be configured to fragment the outer portion and may allow one or more fragments of the outer portion to be separated from the inner portion. The at least one tear surface of the outer portion may configured to fragment the outer portion into at least four fragments.

The disclosure also includes a medical device comprising a sponge that may include one or more perforated tear surfaces that divide the sponge into a plurality of predefined subunits. The medical device comprising a tube. A distal end of the tube may be coupled to a proximal end of the sponge. Each of the plurality of predefined subunits may be a cube or a rectangular prism. The plurality of predefined subunits may be arranged in a plurality of rows. A proximalmost row of the plurality of rows is longer than the other rows of the plurality of rows.

The disclosure also includes a medical method comprising removing a portion of a sponge along a tear surface of the sponge in order to change the sponge from a first, predefined shape or size to a second, predefined shape or size; inserting the sponge into a body lumen of a patient; and connecting a tube coupled to the sponge to a source of vacuum. The method may further comprise removing a portion of the sponge includes removing an outer portion of the sponge from an inner portion of the sponge and removing a detachable layer of the inner portion of the sponge.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts a distal end of an exemplary medical device.

FIG. 2 depicts a cross section of the distal end of the medical system of FIG. 1.

FIG. 3 depicts a cross section of an alternative distal end of a medical system in a first configuration.

FIG. 4 depicts a cross section of the alternative distal end of FIG. 3 in a second configuration.

FIG. 5 depicts an alternative distal end of a medical device.

FIG. 6 depicts a cross section of the distal end of FIG. 5.

FIG. 7 depicts alternative medical devices contained within a kit.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of +10% in a stated numeric value or range.

Endoluminal vacuum therapy (EVT or EVAC, and referred to herein as EVAC) is a procedure to treat wounds, such as post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a nasogastric tube having a sponge/foam (e.g., vacuum sealed foam) at its distal end. A proximal end of the tube may be connected to a collection container. The foam is placed endoscopically into the perforation, leak, or other wound. In some examples, EVAC includes endoluminal placement of a foam or other like material into the wound (e.g., target) site, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement of the material can also be in other organs reachable via the GI tract (e.g., colon). Negative pressure then is applied.

The foam in the wound, along with the negative pressure, may accelerate healing by encouraging local tissue granulation at a wound site. The foam may be replaced with increasing smaller sizes of foam as the wound heals and closes. Devices and systems suited for EVAC are limited. For example, EVAC typically requires a foam to be replaced every 3 to 5 days to reduce the risk of tissue ingrowth. Furthermore, infection of the wound may occur, which may prolong treatment of the wound. It should be understood that the terms “sponge” and “foam” as used in this disclosure are interchangeable with one another and refer to the same structure.

Aspects of this disclosure include devices and methods for sponges having a customizable or selectable size. The sponge may be attached to a vacuum tube. The sponge may include perforations, allowing a user to tear away portions of the sponge in order to select a size of sponge for delivery into a body lumen of a subject. The user may select a size of sponge that is suitable for a wound being treated.

Reference will now be made in detail to examples of the disclosure described above and illustrated in the accompanying drawings. Wherever possible, the same reference numbers will be used throughout the drawings to refer to the same or like parts.

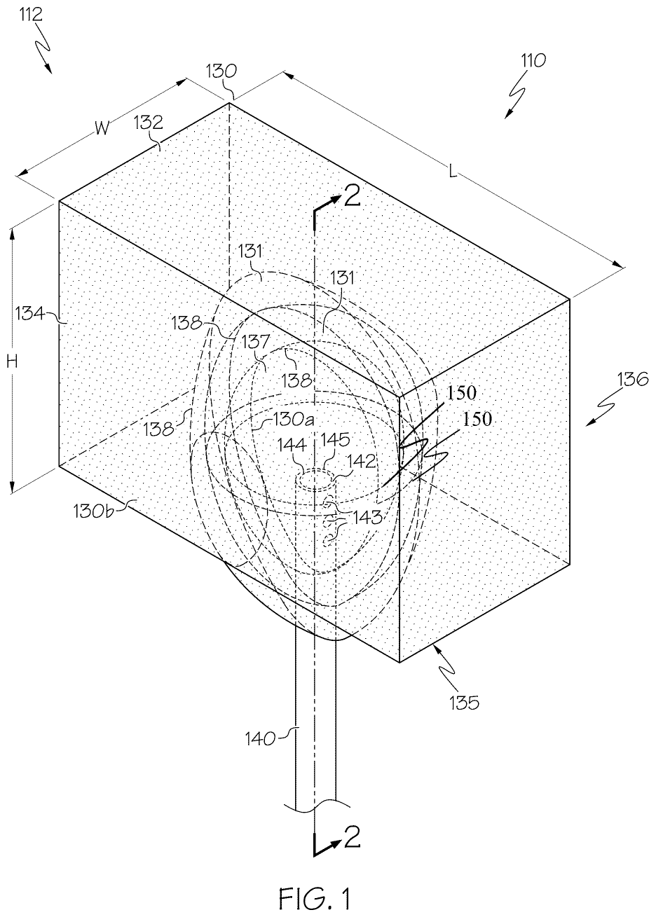

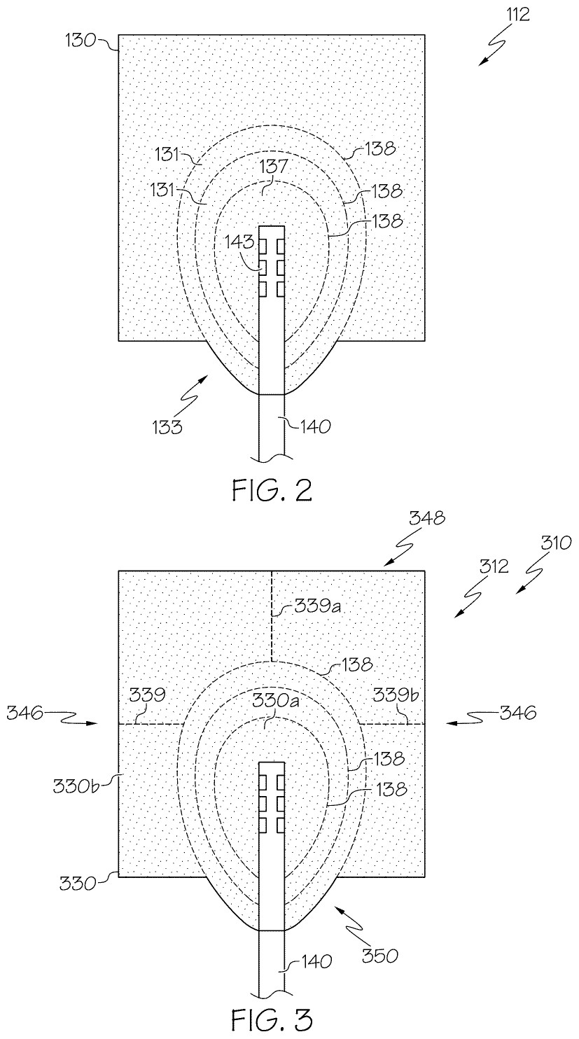

FIGS. 1 and 2 illustrate a distal end 112 of a medical device 110 in accordance with an example of this disclosure. In examples, device 110 may be utilized to perform an EVAC procedure. Device 110 may be inserted into a patient for treatment of wounds using negative pressure via a vacuum. Device 110 generally includes a foam or sponge 130 (or other mesh-like material or porous body) and a vacuum tube 140. Sponge 130 may be formed from Granufoam™ or any other suitable material.

Sponge 130 may be attached to a distal end 141 of vacuum tube 140. Vacuum tube 140 may include an outer wall 142 defining one or more lumens 144. Lumen 144 may be open at both a proximal end and distal end 141 of vacuum tube 140. Outer wall 142 may include a plurality of holes 143 around a circumference of the distal end 141 of vacuum tube 140 and in fluid communication with lumen 144, which may increase the flow or fluid or material into lumen 144, as disclosed herein. Sponge 130 may cover plurality of holes 143 around the circumference of the distal end 141 of vacuum tube 140. In other words, sponge 130 may surround an entire circumference of vacuum tube 140, and a proximal end of sponge 130 may be proximal of a proximalmost hole of plurality of holes 143.

The distal end 141 of vacuum tube 140 may be attached to sponge 130 via sutures, heat bond, an adhesive, a connector (e.g., a quick-connect connector) or the like. In one example, a recess (not shown) may be provided in sponge 130 to receive the distal end 141 of vacuum tube 140. Vacuum tube 140 may be attached within the recess of sponge 130, which may provide additional structural support between sponge 130 and vacuum tube 140. Alternatively, sponge 130 may be extruded or cast on vacuum tube 140 as described herein.

Sponge 130 may include openings 132 throughout sponge 130. Openings 132 may be any hole, pore, or channel which provide continuous access to interconnecting channels and pores throughout sponge 130. Openings 132 may include different sizes and shapes, and may be selected based on a location of treatment within the body. For example, openings 132 may be spherical, cuboidal, irregular, or any other shape. The size of openings 132 may be approximately 50 μm to approximately 1 mm in diameter.

A proximal end of vacuum tube 140 (not shown) may be connected to a vacuum source (not shown), which may supply a negative pressure to sponge 130 and/or the distal end 141 of vacuum tube 140. For example, a negative pressure of approximately 125 mm Hg, or approximately 2.5 pounds per square inch (PSI), may be supplied to sponge 130. Other suitable amount of negative pressure may be used. This negative pressure may pull fluid, material, and/or other debris into lumen 144 of vacuum tube 140 via openings 132, distal opening 145 of vacuum tube 140, and plurality of holes 143, which may promote healing of a target site.

Sponge 130 may include an inner portion 130a and an outer portion 130b, outer portion 130b is illustrated has having a cuboidal or a rectangular prism shape, but outer portion 130b may be any shape including spherical shape, irregular shape, or the like. Inner portion 130a may be fully contained or partially contained within outer portion 130b.

Sponge 130 may include one or more tear surfaces 138, which may be perforated by any suitable means. For example, tear surfaces 138 may include openings/cuts separated by small pieces of foam, allowing an operator to tear along tear surfaces 138 by pulling at tear surfaces 138. In other aspects, the tear surfaces may be guidelines to guide the scissors or cutting devices of a medical professional when trying to form a desired size and shape of sponge 130. In other words, in such aspects, the tear surfaces may merely be visible lines that are drawn on sponge 130. One or more tear surfaces 138 may facilitate the separation of an exterior surface of inner portion 130a from an interior surface of outer portion 130b. One or more tear surfaces 138 may be configured so that a medical professional may separate one or more tear surfaces 138 to achieve a desired size and/or shape. The one or more tear surfaces 138 may be separated by applying a force in a direction away from the one or more tear surfaces 138. One or more tear surfaces may define a predefined location for removing outer portion 130b from inner portion 130a and may define one or more predefined locations for removing one or more detachable layers 131 of inner portion 130a. Detachable layers 131 may be concentric with each other and with a core 137 of inner portion 130a. Detachable layers 131 may be adjacent to each other and core 137. Detachable layers 131 may define a plurality of sizes of an implantable portion of sponge 130.

Sponge 130 may include a front surface 134 and a back surface 136. The front surface 134 and back surface 136 may be opposite one another. Inner portion 130a may include one or more tear surfaces 138 that may form any shape and/or size and may include end portions located at front surface 134 and back surface 136. One or more tear surfaces 138 may extend from front surface 134 to back surface 136. Tear surfaces 138 may form a boundary between inner portion 130a and outer portion 130b. For example, tear surfaces 138 may form a radially inner boundary of outer portion 130b and a radially outer boundary of inner portion 130a. When viewed in the direction of front surface 134 to back surface 136, detachable layers 131 may have a relatively uniform shape from front surface 134 to back surface 136, however the sizes of the detachable layers may increase nearing a center of sponge 130 and decrease nearing front surface 134 and back surface 136. Tear surfaces 138 may be similar shapes to one another and may be nested in one another.

The exemplary embodiment shown in FIGS. 1 and 2 depicts one or more tear surfaces 138 of inner portion 130a forming a match head/balloon-like shape. One or more tear surfaces 138 may include end portions located at front surface 134 and back surface 136 so that each layer of one or more tear surfaces 138 is accessible to the medical professional. In some examples, outer portion 130b may be omitted. While one or more tear surfaces 138 of inner portion 130a are shown forming a matchhead/balloon-like shape, it should be understood that the one or more tear surfaces 138 of inner portion 130a may be any shape (e.g. a cylindrical shape or prismatic shape).

In one or more embodiments, one or more tear surfaces 138 may extend to a distal surface of sponge 130. One or more tear surface 138 may extend to one or more side surfaces of sponge 130.

As seen in FIGS. 1 and 2, the inner portion 130a of sponge 130 may include one or more detachable layers 131. One or more tear surfaces 138 may define the boundaries between one or more detachable layers 131 and/or may form the one or more detachable layers 131. One or more detachable layers 131 may be the same shape as the shape formed between tear surfaces 138. One or more detachable layers 131 may each be the same shape but may be of differing sizes. In other words, tear surfaces 138 may be similarly shaped. In some examples, detachable layers 131 may be concentric with one another. In some examples, tear surfaces 138 may define an entirety of an outer surface of a respective detachable layer 131. In other words, tear surfaces 138 may define a boundary between adjacent detachable layers 131. Tear surfaces 138 may be a radially inward boundary of an outer detachable layer 131 and a radially outward boundary of an inner detachable layer. In some examples, each detachable layer 131 forms a smooth, continuous outer surface.

In aspects, a shape and/or size of sponge 130 may be altered by removing one or more of detachable layers 131. For example, in a first configuration, an outer surface of outer portion 130b may form an outer surface of sponge 130. In a second configuration, outer portion 130b may have been removed, such that an outermost of detachable layers 131 forms the outer surface of sponge 130. In a third configuration, the outermost of detachable layers 131 may have been removed, such that a second-most outer of detachable layers 131 forms the outer surface of sponge 130. In the second configuration, the outer surface of sponge 130 may be completely different than the outer surface of sponge 130 in the third configuration. In other words, portions forming the outer surface in the third configuration may be entirely within the sponge (not exposed) in the second configuration.

One or more detachable layers may each include one or more fragmentation tear surfaces 150. One or more fragmentation tear surfaces 150 may extend from an outer surface to an inner surface of the each detachable layer 131 and may extend horizontally and/or vertically along each detachable layer 131. The one or more fragmentation tear surfaces 150 may allow a medical professional to more easily tear away a detachable layer from either another detachable layer or core 137. It should be understood that any detachable layer 131 may have any number of fragmentation tear surfaces 150. Fragmentation tear surfaces 150 may allow a detachable layer to be divide up into a number of fragments. It should be understood that each fragment may be sutured to tube 140 to prevent the fragments from unwantedly detaching during a procedure.

Inner portion 130a of exemplary sponge 130 shown in FIGS. 1 and 2 includes two tear surfaces 138 yielding two detachable layers 131 of inner portion 130a. However, any suitable number of tear surfaces 138 may be present, creating any suitable number of detachable layers 131. By removing one or more of the detachable layer(s) 131, the medical professional may decrease the size of inner portion 130a of sponge 130 which may be desirable in circumstances where the full size of inner portion 130a (i.e. without any detachable layer(s) 131 being removed) would be too large to be inserted into a target site. In other words, tear surface(s) 138 may allow a user to remove detachable layers 131 in order to customize a size of inner portion 130a, which may be inserted into treatment site. Inner portion 130a may include inner core 137 that is radially inward of all of the detachable layers 131. Inner core 137 may be the smallest possible size of sponge 130. Inner core 137 may be sutured to tube 140, extruded on tube 140, or otherwise fixedly attached to tube 140.

As shown in FIGS. 1 and 2, a proximal portion 133 of inner portion 130a may extend proximally beyond a proximal facing surface 135 of outer portion 130b, the proximal portion 133 may include proximal portions of one or more detachable layers 131. A portion of proximal portion 133 may taper to tube 140. A portion of at least one or of the one or more detachable layers 131 may contact tube 140.

In some examples, only core 137 of inner portion 130a may be sutured to tube 140, and detachable layers 131 may not be sutured to tube 140. Alternatively, one or more detachable layers 131 may be sutured or otherwise removably attached to tube 140. One or more detachable layers 131 may be unsutured or unattached from tube 140 by a user (e.g., a medical professional) prior to using device 110 in a medical procedure. In some aspects, it may be desirable for one or more layers 131 may be sutured or otherwise removably attached to tube 140 to prevent one or more layers 131 from unwantedly detaching from the device 110 while device 110 is positioned within a patient's body.

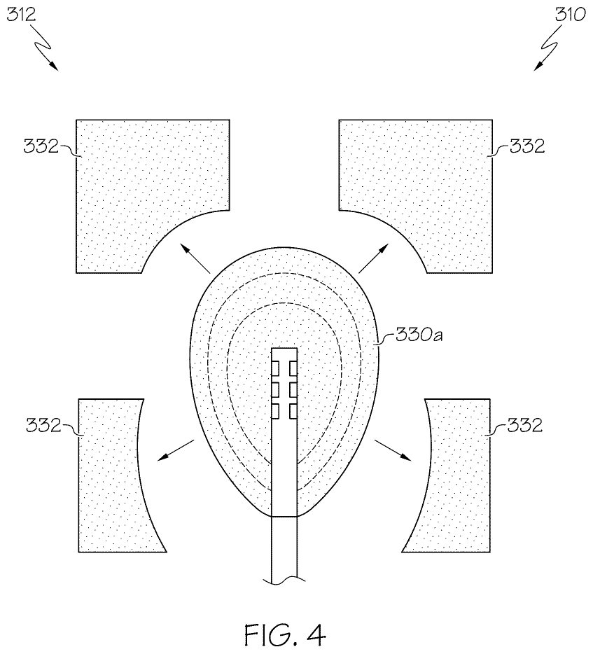

FIGS. 3-4 depicts an alternative distal end 312 of a medical device 310 with an alternative sponge 330. FIG. 3 shows distal end 312 in a first configuration, and FIG. 4 shows distal end 312 in a second configuration. Sponge 330 may have any of the features of sponge 130 or any of the other sponges described in this disclosure. Sponge 330 may be attached or fixed to a distal end 141 of the vacuum tube 140, in any of the manners described above. Sponge 330 may include two side surfaces 346, a top surface 348, and a bottom surface 350. Sponge 330 may also include a front surface and a back surface (not shown in the cross-sectional view of FIG. 3 because they are into or out of the page from the cross-sectional view).

Sponge 330 may include an inner portion 330a and an outer portion 330b. Outer portion 330b may include one or more tear surfaces 339. In the first configuration of FIG. 3, a first tear surface 339a may extend from an outer surface of inner portion 330a to top surface 348. Outside of inner portion 330a, first tear surface 339a may extend between the front surface and the back surface of outer portion 330b. In front of and behind inner portion 330a, first tear surface 339a may extend from top surface 348 to bottom surface 350. First tear surface 339a may be approximately planar and may extend into and out of the page and along a longitudinal axis of device 310 (up and down in FIG. 3). In other words, first tear surface 339a may divide outer portion 330b approximately in half (e.g., into left and right halves), along a central longitudinal axis of device 310.

A second tear surface 339b may extend from the outer surface of inner portion 330a to the two side surfaces 346 and to the front and back surfaces. In other words, second tear surface 339b may extend around a perimeter of inner portion 330a and may extend to side surface 346 and front surface and back surface. Second tear surface 339b may be approximately planar. For example, second tear surface 339b may be approximately perpendicular to a longitudinal axis of device 310. Second tear surface 339b may divide outer portion 330b approximately in half (e.g. into top and bottom halves).

Tear surfaces 339 of outer portion 330b and tear surfaces 138 of sponge 330 may allow for the medical professional to more easily remove outer portion 330b from inner portion 330a. Outer portion 330b may include any number of tear surfaces 138. FIG. 4 shows outer portion 330b being removed from inner portion 330a via one or more tear surfaces 138 and shows outer portion 330b being fragmented into pieces 332 via one or more tear surfaces 339a, 339b. Pieces 332 may be discarded. In the example shown in FIG. 4, outer portion 330b may be fragmented into four pieces 332 (pieces 332 may also be referred to within this disclosure as fragments).

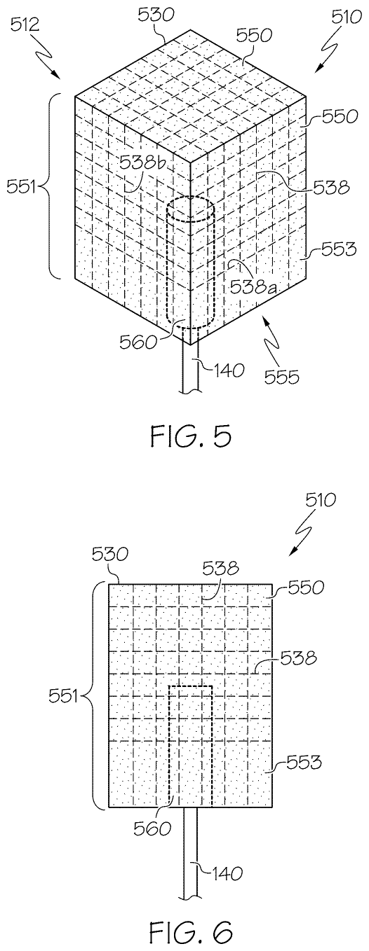

FIGS. 5-6 depict an alternative distal end 512 of a medical device 510 with an alternative sponge 530. Sponge 530 may include any of the features of sponges 130, 330, or any of the sponges described within this disclosure. Sponge 530 may be attached or fixed to the distal end of a vacuum tube 140 via any manner or mechanism described within this disclosure or otherwise known to one skilled in the art. Sponge 530 may include one or more tear surfaces 538. The one or more tear surfaces 538 may be arranged so as to divide sponge 530 into a plurality of predefined subunits 550. For example, tear surfaces 538 may form a grid pattern throughout sponge 530. Plurality of subunits 550 may be cubes, cuboids, and rectangular prisms, and/or combinations thereof. However, plurality of subunits 550 are not limited to the aforementioned shapes and may be any three-dimensional shape. Sponge 530 may include an inner core 560. Inner core 560 may be sutured or otherwise affixed to the distal end of tube 140. Inner core 560 may not include any tear surfaces. In some aspects, inner core 560 may have any of the features of inner portion 130a. In some aspects, one or more of the plurality of subunits 550 may be sutured to inner core 560 and/or tube 140. Inner core 560 may be a cylindrical shape, however inner core 560 may be any shape.

In some examples, sponge 530 may include a plurality of horizontal tear surfaces 538a that extend approximately perpendicularly to a central longitudinal axis of device 510. Horizontal tear surfaces 538a may be approximately planar and may be approximately parallel to one another. Horizontal tear surfaces 538a may form one or more rows of subunits 551 A proximalmost row 553 of the one or more rows of subunits 551 may be a different shape than the other rows of the plurality of subunits 550. For example, as shown in FIGS. 5 and 6, the proximalmost row 553 may be composed of subunits shaped as rectangular prisms, whereas the remaining rows of the one or more rows of subunits 551 formed from subunits shaped as cubes or generally cube-like. Proximalmost row 553 may be longer (in a direction approximately parallel to a longitudinal axis of medical device 510, i.e., the up/down direction of FIG. 5). In other words, a proximalmost horizontal tear surface 538a may be further from a proximal surface 555 of sponge 530 than adjacent horizontal tear surfaces 538a are from one another. In some examples, horizontal tear surfaces 538a may be evenly spaced from one another. In other examples, horizontal tear surfaces 538a may be variably spaced from one another.

Sponge 530 may also include a plurality of vertical tear surfaces 538b. Vertical tear surfaces 538b may be approximately planar and may be approximately parallel to one another. Vertical tear surfaces 538b may extend perpendicularly to horizontal tear surfaces 538a and may be parallel to the central longitudinal axis of medical device 510. In some examples, vertical tear surfaces 538b may be evenly spaced from one another. In other examples, vertical tear surfaces 538b may be variably spaced from one another. In general, tear surfaces 538 may be arranged so that plurality of subunits 550 are arranged in a generally grid-like 3D shape.

Subunits 550 of sponge 530 provide significant benefits to medical professionals utilizing device 510 that incorporates sponge 530. One or more tear surfaces 138 may be configured to facilitate precise removal of one or more subunits 550 from the plurality of subunits 550 allowing a medical professional to quickly and precisely sculpt a desired shape of sponge 530 that is most advantageous based on the circumstances known to the medical professional, such as the shape or size of the target site, the location of the target site, amount of leakage or fluids at the target site, features of the surrounding location of the target site, and other relevant considerations known to those skilled in the art. For example, a medical professional may remove one or more subunits of plurality of subunits 550 to achieve a shape similar to the balloon show in FIGS. 1-4 or any alternative shape. Sponge 530 may be sculpted into a desired shape prior to the insertion of device 110 into the orifice or bodily lumen of the patient. In some aspects, instead of/or in addition to including tear surfaces 538, sponge 530 may include a one or more guidelines arranged on the outer surfaces of sponge 530. The one or more guidelines may be arranged in a similar grid as tear surfaces 538.

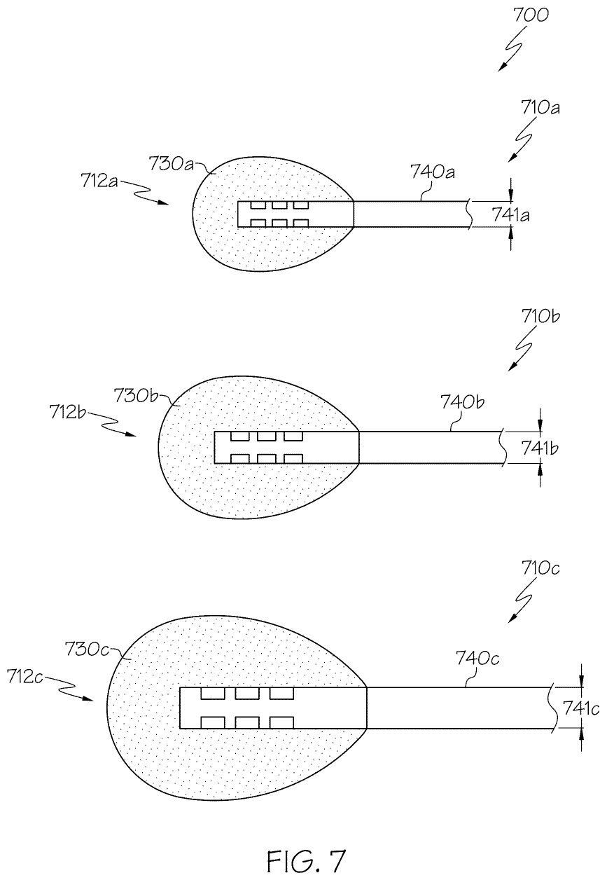

FIG. 7 depicts a kit 700. Kit 700 may include a plurality of medical devices 710a, 710b, 710c, which may be packaged together in a single package. Distal ends 712a, 712b, 712cc of medical devices 710a, 710b, 710c, respectively, may each include a tube and a sponge, described further below, which that may have any of the features as any of the other tubes and sponges described in this disclosure. Medical devices 710a, 710b, 710c may have sponges 730a, 730b, 730c, respectively, of different sizes. A user may select which medical device 710a, 710b, 710c is appropriate for a particular patient.

Distal end 712a of device 710a may include a tube 740a and a sponge 730a. Tube 740a may be received within and secured to sponge 730a, as described above for other sponges and tubes. Tube 740a may have a diameter 741a, which may have any suitable size (e.g., approximately 10 French or any other suitable size). In some aspects, tube 740a may range in size from 7 French to 15 French. As shown in FIG. 7, sponge 730a may be balloon/match head shaped; however, sponge 730a may be any shape (e.g., cylindrical, spherical, rectangular prism shaped, etc.).

Distal end 712b of device 710a may include a tube 740b and a sponge 730b. Tube 740b may be received within and secured to sponge 730b, as described above for other sponges and tubes. Tube 740b may include a diameter 741b, which may have any suitable size (e.g., any size between 12 French and 14 French or any other suitable size). Diameter 741b may be larger than diameter 741a, in some examples. Alternatively, diameter 741b and diameter 741a may be approximately the same size. Sponge may be balloon/match head shaped, as shown in FIG. 7. However, sponge 730b may be any suitable shape, such as any of the shapes described above for sponge 730a. A volume/size of sponge 730b may be larger than a volume/size of sponge 730a. Sponge 730b may have the same size as sponge 730a or may have a different shape.

Distal end 712c of device 710a may include a tube 740c and sponge 730c. Tube 740c may be received within sponge 730c. Tube 740c may include a diameter 741c, which may have any suitable size (e.g. any size between 16 French and 18 French or any other suitable size.) Diameter 741c may be larger than diameter 741b and diameter 741a. Alternatively, all diameters 741a, 741b, 741c may all be the same size. Sponge may be balloon/match head shaped, as shown in FIG. 7. However, sponge 730c may be any suitable shape, such a any of the shapes described above for sponge 730a and/or 730b. A volume/size of sponge 730b may be larger than a volume/size of sponge 730a. Sponge 730b may have the same size as sponge 730a or may have a different shape.

As discussed above, devices 710a, 710b, 710c may be packaged in a kit 700. For example, the kit may include one of each distal end of devices 710a, 710b, 710c as described above. It should be understood that tubes 740a, 740b, and 740c and sponges 730a, 730b, and 730c may be interchangeable with each other; for example, tube 740b may be connected to sponge 730c. Further, the tubes 740a, 740b, 740c may be removably attachable with their respective sponges 730a, 730b, 730c and reattached to a different one of sponges 730a, 730b, 730c (e.g., via quick-connect connectors). Alternatively, in some embodiments, each of tubes 740a, 740b, 740c may be the same diameter while each of sponges 730a, 730b, and 730c may be of differing volumes/sizes and may be of differing shapes. In other embodiments, each of tubes 740a, 740b, 740c may be of differing diameters as discussed above but sponges 730a, 730b, and 730c may be of the same volume and/or same or differing shapes.

In some embodiments, sponges 730a, 730b, 730c included in kit 700 may have any of the features of any of the sponges described within this disclosure. Kit 700 may include one or more device storage spaces (not shown), each of the one or more device storage spaces may be configured to receive one or more of devices 710a, 710b, 710c. The one or more device storage spaces may be substantially the same shape as one or more of devices 710a, 710b, 710c in order to receive said device. It should be understood that the one or more device storage spaces may be formed in an appropriate medium such as foam or plastic or other suitable material used in connection with the storage of medical devices.

In one or more embodiments, packaging the above-described devices 710a, 710b, 710c in a kit may be advantageous for medical professional as the medical professional may be enable to quickly customize and combine the necessary size of the tube with the necessary size and/or shape of the sponge to assemble a distal end appropriate for a particular target site. Further, packaging the above-described distal ends in a kit may enable the medical professional to easily reduce/increase the size of tube and size of the sponge based on the condition of the target site, for example if the target site is healing and decreasing in size the medical professional may want to decrease the size of the tube and/or decrease the size of the sponge. Unused devices 710a, 710b, 710c and their components (e.g. sponges 730a, 730b, 730c and tubes 740a, 740b, 740c) may be discarded or used in a future procedure, the future procedure may require the unused device 710a, 710b, 710c from a previous procedure, reducing medical waste. In some embodiments, Kit 700 may include the devices 710a, 710b, 710c but the devices and their components are all larger sizes. For example, the kit may include sponges and tubes of larger sizes and may be assembled for adult-sized patients. In some embodiments, kit 700 may include devices 710a, 710b, 710c but the devices and their components are all smaller sizes. For example, kit 700 may include sponges and tubes of small sizes and may be assembled for child-sized patients. It should be understood that kit 700 may be assembled with medical devices 710a, 710b, 710c, that have desirable features for certain situations. For example, a kit may be assembled for larger wound cavities at the target site by including medical devices with larger sponges and/or larger tubes. It should be understood that the kit may include any number of sponges and any number of tubes. It should also be understood that sponges 730a, 730b, and 730c may have any of the features of any of the sponges described within this disclosure.

Although the sponges above are described as being fixedly attached to tubes, it will be appreciated that the sponges may be removably coupled to tubes via a connector (e.g., a quick-connect connector). Such a connector may allow for exchange of sponges during a course of treatment to accommodate a changing wound size.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a sponge, wherein the sponge includes a plurality of perforations that form one or more tear surfaces, wherein the one or more tear surfaces define a predefined location for removing a first portion of the sponge from a second portion of the sponge, and wherein the one or more tear surfaces form a boundary between a radially inner surface of the first portion and a radially outer surface of the second portion; and
a tube coupled to a proximal portion of the sponge.

2. The medical device of claim 1, wherein the sponge includes an outer portion and an inner portion, wherein the one or more tear surfaces separate the outer portion from the inner portion, and wherein the outer portion has a different shape from the inner portion.

3. The medical device of claim 2, wherein the inner portion includes at least one of the one or more tear surfaces.

4. The medical device of claim 3, wherein the at least one of the one or more tear surfaces of the inner portion forms one or more detachable layers of the inner portion.

5. The medical device of claim 4, wherein the one or more detachable layers include two detachable layers, and wherein an outer surface of a first detachable layer of the two detachable layers is adjacent to an inner surface of a second detachable layer of the two detachable layers.

6. The medical device of claim 5, wherein the first detachable layer is concentric with the second detachable layer.

7. The medical device of claim 5, wherein the inner portion further includes a core, wherein the first detachable layer is adjacent to the core.

8. The medical device of claim 4, wherein the outer portion includes a proximal surface, and wherein a proximal portion of at least one of the one or more detachable layers extends proximally of the proximal surface of the outer portion.

9. The medical device of claim 8, wherein the proximal portion of the at least one of the one or more detachable layers contacts the tube.

10. The medical device of claim 2, wherein the outer portion has a first surface and a second surface, and wherein at least one of the one or more tear surfaces extends from the first surface to the second surface between the inner portion and the outer portion.

11. The medical device of claim 10, wherein the outer portion includes at least one of the one or more tear surfaces.

12. The medical device of claim 11, wherein the at least one tear surface of the outer portion is configured to fragment the outer portion and allow one or more fragments of the outer portion to be separated from the inner portion.

13. The medical device of claim 12, where the at least one tear surface of the outer portion is configured to fragment the outer portion into at least four fragments.

14. The medical device of claim 1, wherein the one or more tear surfaces are configured to define a plurality of sizes of an implantable portion of the sponge.

15. The medical device of claim 1, wherein the one or more tear surfaces define a grid.

16. A medical device comprising:
a sponge including one or more perforated tear surfaces that divide the sponge into a plurality of predefined subunits; and
a tube, wherein a distal end of the tube is coupled to a proximal end of the sponge.

17. The medical device of claim 16, wherein each of the plurality of predefined subunits is a cube or a rectangular prism.

18. The medical device of claim 16, wherein the plurality of predefined subunits are arranged in a plurality of rows, wherein a proximalmost row of the plurality of rows is longer than the other rows of the plurality of rows.

19. A medical method comprising:
removing a portion of a sponge along a tear surface of the sponge in order to change the sponge from a first, predefined shape or size to a second, predefined shape or size;
inserting the sponge into a body lumen of a patient; and
connecting a tube coupled to the sponge to a source of vacuum.

20. The medical method of claim 19, wherein removing a portion of the sponge includes removing an outer portion of the sponge from an inner portion of the sponge and removing a detachable layer of the inner portion of the sponge.
